(19) United States
(12) Patent Application Publication
(10) Pub. No.: US 2010/0075417 A1
(43) Pub. Date: Mar. 25, 2010

(54) Methods and devices for isolating embryonic stem cells

(75) Inventors: Michael Cohen, Baltimore, MD (US); Xiangcan Zhan, Baltimore, MD (US); Michael J. Shamblott, Boltimore, MD (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(73) Assignees: Proteonomix, Inc., Mountainside, NJ (US); The Johns Hopkins University, Baltimore, MD (US)

(21) Appl. No.: 12/454,263

(22) Filed: May 14, 2009

Related U.S. Application Data
(60) Provisional application No. 61/071,734, filed on May 14, 2008.

(51) Int. Cl. C12N 5/0735; C12M 1/12

(52) U.S. Cl. 435/366; 435/325; 435/297.1

(57) ABSTRACT

Methods, devices and kits are provided for isolating or enriching multicellular embryonic stem (ES) cell colonies from a mixture of multicellular ES cell colonies and single ES cells present in a cellular suspension, by utilizing a filtration matrix that selectively excludes passage of multicellular ES cell colonies. Isolated or enriched multicellular ES cell colonies can be used for propagating pluripotent ES cells.

### BACKGROUND

Embryonic stem (ES) cells are derived from the inner cell mass of preimplantation embryos. ES cells have been derived from several species, including mouse, pig, chicken, and human. ES cells are pluripotent and are capable of differentiating into cells derived from all three embryonic germ layers.

Following routine trypsinization or other methods of disaggregation, human ES cells obtained from cultures are present as single cells and as multicellular colonies. Although single cells are desirable under certain circumstances such as for clonal isolation following genetic engineering, routine passaging of single ES can more readily result in aneuploid cell cultures than if ES cells are cultured as multicellular colonies.

### SUMMARY

In a first embodiment, a method is provided for isolating or enriching multicellular ES cell colonies from a mixture of multicellular ES cell colonies and single ES cells present in a cellular suspension comprising the steps of (a) providing a filtration matrix through which single ES cells but not multicellular ES cell colonies can pass from a first side to a second side; (b) contacting the cellular suspension with the first side of the filtration matrix; (c) passing the cellular suspension from the first side to the second side of the filtration matrix; and (d) collecting the multicellular ES cell colonies from the first side of the filtration matrix, thereby isolating or enriching multicellular ES cell colonies therefrom.

In another embodiment, the filtration matrix comprises polyester, polyamide, aramid, acrylic, or PTFE. In still another embodiment, the filtration matrix has a porosity of about 20 microns to about 30 microns. In yet another embodiment, the passing is via centrifugation, application of positive pressure to the first side of the filtration matrix, application of negative pressure to the second side of the filtration matrix, or gravity filtration.

In a further embodiment of the aforementioned method, multicellular ES cell colonies can be collected after following the aforementioned methods by carrying out the additional steps of (a) discarding the single ES cells from the second side of the filtration matrix; (b) passing a liquid medium from the second side to the first side of the filtration matrix, whereby multicellular ES cell colonies retained on the first side are suspended in the liquid medium; and then (c) collecting the liquid medium.

In still another embodiment, the passing can be accomplished by a combination of lateral flow along the first side of the filtration matrix and transverse flow from the first side to the second side of the filtration matrix. In a further embodiment, the flow is continuous or interrupted. In yet another embodiment, the single ES cells pass transversely from the first side to the second side of the filtration matrix, while multicellular ES cell colonies are enriched in the cellular suspension during lateral flow along the first side of the filtration matrix.

In another embodiment, the filtration medium is cylindrical, and the passing is along the interior of the cylindrical filtration medium, or along the exterior of the cylindrical filtration medium.

In another embodiment, a method is provided for cultivating euploid human ES cells comprising the steps of (a) treating a human ES cell culture to detach adherent single ES cells and multicellular ES cell colonies therefrom; (b) collecting a cellular suspension comprising multicellular ES cell colonies and single ES cells from step (a); (c) isolating multicellular ES cell colonies from the suspension by any of the embodiments described hereinabove; and (d) inoculating the multicellular ES cell colonies from step (c) into ES cell culture medium. In another embodiment, treating is trypsinizing.

In another embodiment, device is provided for isolating or enriching multicellular ES cell colonies from a mixture of multicellular ES cell colonies and single ES cells present in a cellular suspension comprising (a) a filtration matrix through which single ES cells but not multicellular ES cell colonies can pass transversely from a first side to a second side; (b) means for passing the cellular suspension at least transversely through the filtration matrix; and (c) means for collecting the isolated or enriched multicellular ES cell colonies from the first side of the filtration matrix.

In another embodiment, the filtration matrix of the device comprises polyester, polyamide, aramid, acrylic, or PTFE. In still another embodiment, the filtration matrix of the device has a porosity of about 20 microns to about 30 microns. In yet another embodiment, the filtration matrix of the device is planar or cylindrical. In a further embodiment, the means for passing comprises a reservoir contiguous with the first side of the filtration matrix and means for collecting comprises a reservoir contiguous with the second side of the filtration matrix. In yet another embodiment, the device further comprises means for passing the cellular suspension laterally along the first side of the filtration matrix. In another embodiment, the filtration matrix is cylindrical and the first side of the filtration matrix is disposed laterally in the direction of passing of the cellular suspension. In another embodiment, the first side of the filtration matrix is interior to the cylinder, whereby multiple ES cell colonies are enriched in the cellular suspension passing through the inside of the cylinder, and single ES cells pass transversely across the filtration matrix to the exterior of the cylinder. In yet another embodiment, the first side of the filtration matrix is exterior to the cylinder, whereby multiple ES cell colonies are enriched in the cellular suspension passing along the outside of the cylinder, and single ES cells pass transversely across the filtration matrix to the interior of the cylinder.

In another embodiment, a kit for isolating or enriching multicellular ES cell colonies from a mixture of multicellular ES cell colonies and single ES cells is provided comprising any device as described above, and instructions for use of the kit.

In another embodiment, a system for isolating or enriching multicellular ES cell colonies or for cultivating euploid human ES cells is provided comprising a device for separating multicellular ES cell colonies from single ES cells, in accordance with the embodiments above.

### BRIEF DESCRIPTION OF THE DRAWINGS

FIG. 1 shows a device for separating single ES cells from multicellular ES cell colonies; and

FIG. 2 shows a continuous flow device for separating single ES cells from multicellular ES cell colonies, whereby single ES cells are eliminated from the main flow stream lateral to the filtration matrix by selective filtration across the filtration matrix, thus enriching the main flow stream for multicellular ES cell colonies.

### DETAILED DESCRIPTION OF THE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

As noted above, it is desirable when cultivating human ES cells to utilize multicellular ES cell colonies rather than single ES cells, since the latter may be aneuploid and would give rise to cells potentially not retaining stem cell-like pluripotent characteristics, i.e., capability of differentiating into numerous terminal cell types. On the other hand, multicellular ES cell colonies obtained from ES cell cultures contain euploid cells, and are preferably used for propagating ES cell cultures. Thus, in one embodiment, a method is provided for collecting from or enriching in a cellular suspension that comprises a mixture of single ES cells and multicellular ES cell colonies, only multicellular ES cell colonies. In one embodiment, the method comprises the steps of

a. providing a filtration matrix through which single ES cells but not multicellular ES cell colonies can pass from a first side to a second side;

b. contacting the cellular suspension with the first side of the filtration matrix;

c. passing the cellular suspension from the first side to the second side of the filtration matrix, such that multicellular ES cell colonies are retained on the first side of the filtration matrix, and single ES cells pass through the filtration matrix to the second side; and

d. collecting the multicellular ES cell colonies from the first side of the filtration matrix, thereby isolating or enriching multicellular ES cell colonies therefrom. In another embodiment, the filtration matrix can be washed or rinsed with a solution such as culture medium or saline to force any single ES cells remaining on the first side to the second side. Multicellular ES cell colonies can then be collected from the first side.

The filtration matrix can be made from polyester (e.g., DACRON), polyamide (e.g., nylon), aramid (e.g., NOMEX), acrylic (e.g., ORLON, DRALON), polytetrafluoroethylene (PTFE, or TEFLON), by way of non-limiting examples, or any other medium, matrix or filtration material which achieves the desired selective filtration purpose. In one embodiment, the filtration matrix is a fabric. In one embodiment, the filtration matrix is resistant to temperatures required for heat sterilization, such that the preparation or manufacture of a device for carrying out the methods described here can be readily achieved and sterility of the cells not compromised. Alternatively, materials may be selected on the basis of price, ease of manufacture or cell adhesion properties, and sterilized using chemical methods, such as disinfectant solutions or exposure to ethylene oxide gas (ETO), radiation, etc.

The pore size of the filtration matrix is selected such that multicellular ES cell colonies are retained by, or do not pass across, the filtration matrix under the conditions selected for carrying out the filtration or separation process, but single ES cells do pass across. For most procedures, such as gravity or pressure/vacuum filtration, the pore size optimally is between about 20 and about 30 microns. In other embodiments, such as the continuous flow system described below, or higher pressure systems, a different porosity can be employed to achieve the same desired goals. In another embodiment, the porosity is greater than 20-30 microns.

The filtration matrix may be symmetric or asymmetric, i.e., it may provide the filtration properties set forth in the embodiments herein by passage in either direction (symmetric), or only in one direction (asymmetric). As described in the embodiments herein, the direction of transverse flow (across) the filtration matrix is from the first side to the second side, wherein multicellular ES cell colonies are retained on or fail to traverse the first side, and single ES cells can pass transversely from (i.e., across or through) the first side to the second side. In other embodiments herein, the filtration matrix is asymmetric.

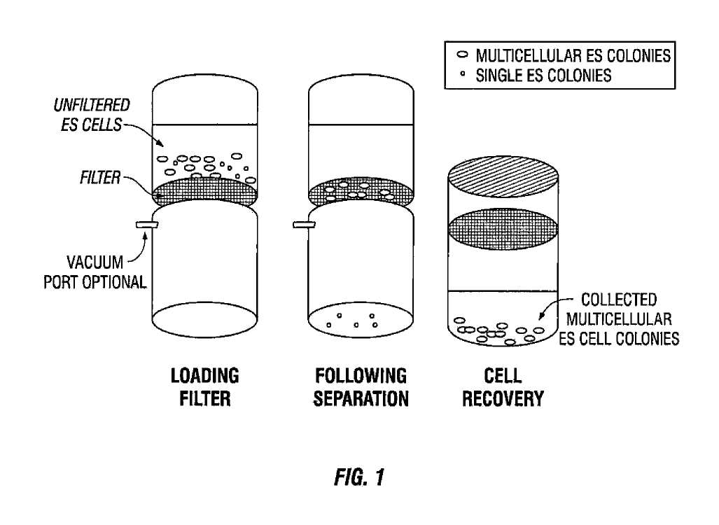

In one embodiment shown in FIG. 1, a device is provided such that a planar (flat) filtration matrix is interposed between an upper reservoir and a lower reservoir. A suspension of culture medium or other solution containing multicellular and single ES cells is placed in the upper reservoir in contact with the first side of the filtration matrix, and the suspension caused to pass through the filtration matrix by using pressure, vacuum or by centrifugal force produced by a pressure system, vacuum system or centrifuge, respectively. Such application of force initiates passage of the suspension from the first side to the second side of the filtration matrix, whereby the multicellular ES cell colonies do not pass across but are retained on the first side, yet single ES cells pass across into the lower reservoir. Similar devices used for other purposes such as sterilizing culture medium are commonly found in tissue culture facilities. Additional medium or an ES cell-compatible solution can be added to the upper reservoir and caused to pass across the filter to carry any residual single ES cells retained on the first side of the filter to pass to the second side. Following the filtration step and removal of the single ES cells, the aforementioned system can be inverted or in some other way manipulated so that the multicellular ES cell colonies cells retained on the first side of the filtration matrix can be efficiently collected. In one embodiment, the device is inverted and using centrifugal force, retained colonies are caused to enter a vessel containing growth media. In another embodiment, the device is inverted and medium or another solution is placed in the lower reservoir, and caused to pass from the second side to the first side (by pressure, vacuum or centrifugation, by way of non-limiting example), thus suspending multicellular ES cell colonies from the first side into the solution. The suspension containing isolated or enriched multicellular ES cell colonies can be used for propagation or any other purpose.

In other embodiments of the planar filtration matrix as described above, various alternate arrangements of the filtration matrix can be provided. In one embodiment, a continuously or intermittently operating in-line filter is provided, where the mixed cellular suspension is pumped through a filtration matrix as described herein. Multicellular ES cell colonies are retained on the filtration matrix and single ES cells pass through. Once the separation is achieved, the in-line filter can be flushed in the same direction with medium to remove any single ES cells from the line and first side of the filtration matrix, then medium pumped in the reverse direction to resuspend and wash the retained multicellular ES cell colonies from the first side of the filtration matrix into the line, from which they are collected. Such an in-line system can be operated manually, semi-manually or automatedly for preparing large quantities of multicellular ES cell colonies for cultivation or any other purpose.

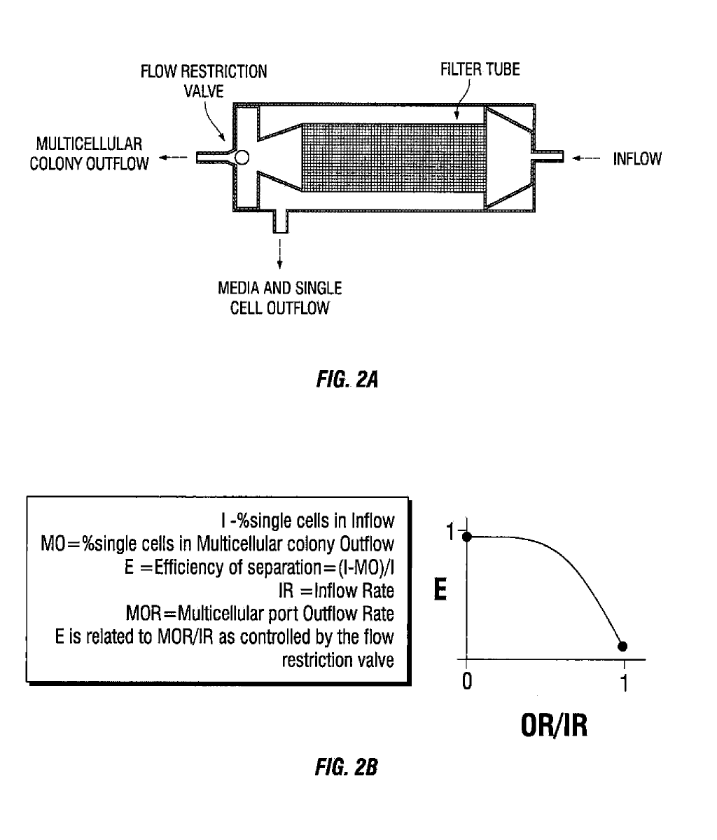

In another embodiment, shown in FIG. 2, a filtration matrix is provided in a lateral (i.e. same) orientation to the direction of flow of the cellular suspension containing a mixture of multicellular ES cell colonies and single ES cells. In the non-limiting example shown, the filtration matrix is cylindrical and the lateral flow is along the interior to the cylinder. Passage of the cellular suspension lateral to the filter (i.e., longitudinally though the cylinder) is accompanied by an extent of flow transverse to, i.e., across, the filtration matrix, whereby single ES cells are pushed out of the flow stream and through the filtration matrix. Depending on the pressure, flow, and other conditions, as the cellular suspension travels laterally along the filtration matrix, elimination of single ES cells provides an enrichment of the flow stream in multicellular ES cell colonies, which after passage through the cylinder can be used for propagation or any other purpose. FIG. 2 shows the parameters of operation of this embodiment contributing to the efficiency of separation.

Thus, in one embodiment, the passing is by a combination of lateral flow along the first side of the filtration matrix and transverse flow from the first side to the second side of the filtration matrix. In another embodiment, the flow is continuous or interrupted. In other embodiments, the passing is along the interior of the cylindrical filtration medium, or along the exterior of the cylindrical filtration medium.

In other embodiments, a device is provided that achieves the aforementioned purposes. In one embodiment, a device is provided for isolating or enriching multicellular ES cell colonies from a mixture of multicellular ES cell colonies and single ES cells present in a cellular suspension comprising:

a. a filtration matrix through which single ES cells but not multicellular ES cell colonies can pass transversely from a first side to a second side;

b. means for passing the cellular suspension at least transversely through the filtration matrix; and

c. means for collecting the isolated or enriched multicellular ES cell colonies from the first side of the filtration matrix.

The filtration matrix can be made from polyester (e.g., DACRON), polyamide (e.g., nylon), aramid (e.g., NOMEX), acrylic (e.g., ORLON, DRALON), polytetrafluoroethylene (PTFE, or TEFLON), by way of non-limiting examples, or any other medium, matrix or filtration material which achieves the desired selective filtration purpose. In one embodiment, the filtration matrix is a fabric. In one embodiment, the filtration matrix is resistant to temperatures required for heat sterilization, such that the preparation or manufacture of a device for carrying out the methods described here can be readily achieved and sterility of the cells not compromised. Alternatively, materials may be selected on the basis of price, ease of manufacture or cell adhesion properties, and sterilized using chemical methods, such as disinfectant solutions or exposure to ethylene oxide gas (ETO), radiation, etc.

The pore size of the filtration matrix is selected such that multicellular ES cell colonies are retained by, or do not pass across, the filtration matrix under the conditions selected for carrying out the filtration or separation process, but single ES cells do pass across. For most procedures, such as gravity or pressure/vacuum filtration, the pore size optimally is between about 20 and about 30 microns. In other embodiments, such as the continuous flow system described below, or higher pressure systems, a different porosity can be employed to achieve the same desired goals. In another embodiment, the porosity is greater than 20-30 microns.

The filtration matrix may be symmetric or asymmetric, i.e., it may provide the filtration properties set forth in the embodiments herein by passage in either direction (symmetric), or only in one direction (asymmetric). As described in the embodiments herein, the direction of transverse flow (across) the filtration matrix is from the first side to the second side, wherein multicellular ES cell colonies are retained on or fail to traverse the first side, and single ES cells can pass transversely from (i.e., across or through) the first side to the second side. In other embodiments herein, the filtration matrix is asymmetric.

In another embodiment of the device, the filtration matrix is planar or cylindrical. In another embodiment, the means for passing comprises a reservoir contiguous with the first side of a planar filtration matrix and means for collecting comprises a reservoir contiguous with the second side of the planar filtration matrix.

In another embodiment, the filtration matrix is cylindrical and the first side of the filtration matrix is disposed laterally in the direction of passing of the cellular suspension. In another embodiment, the first side of the filtration matrix is interior to the cylinder, whereby multiple ES cell colonies are enriched in the cellular suspension passing through the inside of the cylinder, and single ES cells pass transversely across the filtration matrix to the exterior of the cylinder. In an alternate embodiment, the first side of the filtration matrix is exterior to the cylinder, whereby multiple ES cell colonies are enriched in the cellular suspension passing along the outside of the cylinder, and single ES cells pass transversely across the filtration matrix to the interior of the cylinder.

In other embodiments, kits comprising a device as described above, such as the planar filtration matrix or the cylindrical filtration matrix, are provided, along with instructions for use.

In another embodiment, any of the aforementioned methods or devices can be used in the process of cultivating euploid ES cells. As noted above, it is preferable to use multicellular ES cell colonies rather than single ES cells taken from ES cultures for propagation purposes, since the multicellular ES cell colonies are generally euploid but single ES cells can be aneuploid and not appropriate for routine passaging. The method of cultivating euploid ES cells comprises the steps of:

a. treating a human ES cell culture to detach adherent single ES cells and multicellular ES cell colonies therefrom;

b. collecting a cellular suspension comprising multicellular ES cell colonies and single ES cells from step (a);

c. isolating multicellular ES cell colonies from the suspension by any of the methods described hereinabove or using any device described hereinabove; and

d. inoculating the multicellular ES cell colonies from step (c) into ES cell culture medium.

In one embodiment, detachment is achieved by mechanical dislodging or trypsinization.

The aforementioned methods and devices are provided to improve on the ability to maintain ES cells in culture while retaining the pluripotency thereof, such that ES cells can be used for cellular and other therapies, among other purposes. The therapeutic and other applications of embryonic stem (ES) cells are projected to have a major impact on the future of health care and the treatment of a large number of diseases. While ES cells used in the various embodiments herein may be of any species, typically human ES cells are employed. Various non-limiting uses of the ES cells isolated, enriched or propagated in accordance with the embodiments herein are described below.

Cell-Based Therapies: Transplantation of ES Cells. ES cells or their differentiated progeny can be used in human transplantations in the fetus, newborns, infants, children, and/or adults. One example of this use is therapeutic supplementation of metabolic enzymes for the treatment of autosomal recessive disorders. For example, production of homogentisic acid oxidase by transplanted ES differentiated cells into the liver could be used in the treatment of alkaptonuria (for review of this disorder, see McKusick, Heritable Disorders of Connective Tissue. 4th ed., St. Louis, C. V. Mosby Co., 1972). Likewise, ornithine transcarbamylase expression could be augmented to treat the disease caused by its deficiency. In another example, glucose-6-phosphate dehydrogenase expression could be augmented in erythrocyte precursors or hematopoietic precursors to allow expression in red blood cells in order to treat G6PD deficiency (favism, acute hemolytic anemia).

Treatments of some diseases require addition of a composition or the production of a circulating factor. One example is the production of alpha 1-antitrypsin in plasma to treat a deficiency that causes lung destruction, especially in tobacco smokers. Other examples of providing circulating factors are the production of hormones, growth factors, blood proteins, and homeostatic regulators.

ES cells are used to repair or supplement damaged or degenerating tissues or organs. This may require that the cells are first differentiated in vitro into lineage-restricted stem cells or terminally differentiated cells.

Before implantation or transplantation the ES cell obtained or grown as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This significantly improves renal function in mouse kidney allografts (see, e.g., Coffinan, J. Immunol. 151:425-435, 1993) and allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. As an alternative to a universal donor approach to histocompatibility, genetic manipulation could be used to generate “custom” MHC profiles to match individual needs.

In addition to manipulating MHC expression, for human transplantation, cells and tissues from ES cells can also be manipulated to eliminate or reduce other cell-surface marker molecules that induce tissue/organ graft rejection. All such modifications that reduce or eliminate allogenic (e.g., organ graft) rejection when employing cells, cell lines (or any parts or derivatives thereof) derived from the cells are embodied herein.

Tissue Engineering. Human cells can be used to produce or reconstruct a tissue or organ, including in vitro or vivo regeneration, and engineering of artificial organs or organoids. In one aspect, the ES cells are pre-cultured under conditions that promote generation of a desired differentiated, or restricted, cell lineage. The culture conditions can also be manipulated to generate a specific cell architecture, such as the three-dimensional cellular arrangements and relationships seen in specialized structures, such as neuromuscular junctions and neural synapses, or organs, such as livers, and the like. These conditions can include the use of bioreactor systems to influence the generation of the desired cell type. Bioreactor systems are commonly used in the art of tissue engineering to create artificial tissues and organs. Some bioreactor systems are designed to provide physiological stimuli similar to those found in the natural environments. Others are designed to provide a three-dimensional architecture to develop an organ culture. For example, the compositions (including bioreactors, scaffolds, culture devices, three-dimensional cell culture systems, and the like) and methods described in U.S. Pat. Nos. 6,143,293; 6,121,042; 6,110,487; 6,103,255; 6,080,581; 6,048,721; 6,022,743; 6,022,742; 6,008,049; 6,001,642; 5,989,913; 5,962,325; 5,858,721; 5,843,766; 5,792,603; 5,770,417; 5,763,279; 5,688,687; 5,612,188; 5,571,720; 5,770,417; 5,626,863; 5,523,228; 5,459,069; 5,449,617; 5,424,209; 5,416,022; 5,266,480; 5,223,428; 5,041,138; and 5,032,508; or variations thereof, can be used in conjunction herein.

As discussed above, production of cells, tissues and organs for transplantation may require combinations of genetic modifications, in vitro differentiation, and defined substrate utilization of the cells to generate the desired altered cell phenotype and, if a tissue or organ is to be generated, the necessary three-dimensional architecture required for functionality. For example, a replacement organ may require vasculature to deliver nutrients, remove waste products, and deliver products, as well as specific cell-cell contacts. A diverse cell population will be required to carry out these and other specialized functions, such as the capacity to repopulate by lineage-restricted stem cells.

Further examples of the use of the ES cells and their differentiated derivatives include generation of non-cellular structures such as bone or cartilage replacements.

Human ES cells can also be implanted into the central nervous system (CNS) for the treatment of disease or physical brain injury, such as ischemia or chemical injury; animal models can also be used to test the efficacy of this treatment, e.g., injection of compounds like 60HAD, or, fluid percussion injury can serve as a model for human brain injury. In these animal models, the efficacy of administration of stem cells is determined by the recovery of improvement of injury related deficits, e.g., motor or behavioral deficits. Human ES cells obtained in accordance with the teaching herein can also be implanted into the central nervous system (CNS) for the treatment of amyotropic lateral sclerosis (ALS); animal models can also be used to test the efficacy of this treatment, e.g., the SODI mutant mouse model. Human ES cells can also be implanted into the central nervous system (CNS) for the treatment of Alzheimer's disease; one animal model that can be used to test the efficacy of this treatment is the mutant presenilin I mouse. Human ES cells can also be implanted into the CNS for the treatment of Parkinson's disease, efficacy of this treatment can be assessed using, e.g., the MPTP mouse model.

Human ES cells can also be used to treat diseases of cardiac, skeletal or smooth muscles; cells can be directly injected into or near desired sites. The survival and differential of these cells can be determined by monitoring the expression of appropriate markers, e.g, human muscle-specific gene products (see, e.g., Klug, 1996, supra; Soonpaa, Science 264:98-101, 1994; Klug, Am. J. Physiol. 269:H1913-H1921, 1995; implanting fetal cardiomyocytes and mouse ES-derived cells), for exemplary protocols.

Human ES cells can also be used to treat diseases of the liver or pancreas. Cells can be directly injected into the hepatic duct or the associated vasculature. Similarly, cells could be delivered into the pancreas by direct implantation or by injection into the vasculature. Cells engraft into the liver or pancreatic parenchyma, taking on the functions normally associated with hepatocytes or pancreatic cells, respectively. As with other implantations, cell survival, differentiation and function can be monitored by, e.g., immunohistochemical staining, or PCR, of specific gene products.

Human ES cells can also be used to treat diseases, injuries or other conditions in or related to the eyes. Cells can be directly injected into the retina, optic nerve or other eye structure. In one aspect, cells differentiate into retinal epithelia, nerve cells or other related cell types. As with other engraftments, cell survival, differentiation and function can be monitored by, e.g., immunohistochemical staining, or PCR, of specific gene products.

Human ES cells can also be used to treat vascular diseases or other related conditions by repopulation of the vasculature with, e.g., vascular endothelium, vascular smooth muscle and other related cell types. For example, an injured vein or artery is treated by implantation of ES cells; these cells re-populate the appropriate injured sites in the vasculature. The cells can be implanted/injected into the general circulation, by local (“regional”) injection (e.g., into a specific organ) or by local injection, e.g., into a temporarily isolated region. In an alternative procedure, a reconstructed or a completely new vasculature can be constructed on a biomatrix or in an organotypic culture, as described herein.

Human ES cells can also be used to repopulate bone marrow, e.g., in situations where bone marrow has been ablated, e.g., by irradiation for the treatment of certain cancers. Protocols for these treatments can be optimized using animal models, e.g., in animals whose endogenous bone marrow has been ablated. EBD cells can be injected into the circulatory system or directly into the marrow space of such an animal (e.g., a rodent model). Injection of the human cells would allow for the re-population of bone marrow, as well as engraftment of a wide range of tissues and organs. If the animals are sublethally irradiated, the efficacy of the cells can be monitored by tracking animal survival, as without bone marrow re-population the animal will die. The hematopoietic fate of the injected cells also can be examined by determining the type and amount to human cell colonies in the spleen.

In another aspect, the human ES cells can be used in organotypic co-culture. This system offers the benefits of direct cell application and visualization found in in vitro methods with the complex and physiologically relevant milieu of an in vivo application. In one aspect, a section of tissue or an organ specimen is placed into a specialized culture environment that allows sufficient nutrient access and gas exchange to maintain cellular viability.

In using the human ES cells, or differentiated derivatives thereof, to construct artificial organs or organoids, bioengineered matrices or lattice structures can be populated by single or successive application of these human cells. The matrices can provide structural support and architectural cues for the repopulating cells.

Biosensors and Methods of Screening. ES cells or cell lines and cells, tissues, structures and organs derived from them can be used for toxicological, mutagenic, and/or teratogenic in vitro tests and as biosensors. Thus, engineered cells, tissues and organs for screening methods can replace animal models and form novel human cell-based tests. These systems are useful as extreme environment biosensors. ES cells or cell lines and cells, tissues, structures and organs derived from them can be used to build physiological biosensors; for example, they can be incorporated in known system, as described, e.g., in U.S. Pat. Nos. 6,130,037; 6,129,896; and 6,127,129. These sensors can be implanted bio-electronic devices that function as in vivo monitors of metabolism and other biological functions, or as an interface between human and computer.

A method for identifying a compound that modulates an ES cell function in some way (e.g., modulates differentiation, cell proliferation, production of factors or other proteins, gene expression) using ES cells is also provided. The method includes: (a) incubating components comprising the compound and ES cell(s) sufficient to allow the components to interact; and (b) determining the effect of the compound on the ES cell(s) before and after incubating in the presence of the compound. Compounds that ES cell function include peptides, peptidomimetics, polypeptides, chemical compounds and biologic agents. Differentiation, gene expression, cell membrane permeability, proliferation and the like can be determined by methods commonly used in the art. The term “modulation” refers to inhibition, augmentation, or stimulation of a particular cell function.

ES Cells as Sources of Macromolecules. The ES cells and cell lines can also be used in the biosynthetic production of macromolecules. Non-limiting examples of products that could be produced are blood proteins, hormones, growth factors, cytokines, enzymes, receptors, binding proteins, signal transduction molecules, cell surface antigens, and structural molecules. Factors produced by undifferentiated, differentiating, or differentiated ES cells would closely simulate the subtle folding and secondary processing of native human factors produced in vivo. Biosynthetic production by ES cells and cell lines can also involve genetic manipulation followed by in vitro growth and/or differentiation. Biosynthetic products can be secreted into the growth media or produced intracellularly or contained within the cell membrane, and harvested after cell disruption. Genetic modification of the gene coding for the macromolecule to be biosynthetically produced can be used to alter its characteristics in order to supplement or enhance functionality. In this way, novel enhanced-property macromolecules can be created and pharmaceuticals, diagnostics, or antibodies, used in manufacturing or processing, can be produced. Pharmaceutical, therapeutic, processing, manufacturing or compositional proteins that may be produced in this manner include, e.g., blood proteins (clotting factors VIII and IX, complement factors or components, hemoglobins or other blood proteins and the like); hormones (insulin, growth hormone, thyroid hormone, gonadotrophins, PMSG trophic hormones, prolactin, oxytocin, dopamine, catecholamines and the like); growth factors (EGF, PDGF, NGF, IGF and the like); cytokines (interleukins, CSF, GMCSF, TNF, TGF.alpha., TGF.beta., and the like); enzymes (tissue plasminogen activator, streptokinase, cholesterol biosynthetic or degradative, digestive, steroidogenic, kinases, phosphodiesterases, methylases, de-methylases, dehydrogenases, cellulases, proteases, lipases, phospholipases, aromatase, cytochromes adenylate or guanylate cyclases and the like); hormone or other receptors (LDL, HDL, steroid, protein, peptide, lipid or prostaglandin and the like); binding proteins (steroid binding proteins, growth hormone or growth factor binding proteins and the like); immune system proteins (antibodies, SLA or MHC gene products); antigens (bacterial, parasitic, viral, allergens, and the like); translation or transcription factors, oncoproteins or proto-oncoproteins, milk proteins (caseins, lactalbumins, whey and the like); muscle proteins (myosin, tropomyosin, and the like).

Screens for Culture Media Factors. ES cells are used to optimize the in vitro culture conditions for differentiating the cells. High-throughput screens can be established to assess the effects of media components, exogenous growth factors, and attachment substrates. These substrates include viable cell feeder layers, cell extracts, defined extracellular matrix components, substrates which promote three-dimensional growth such as methylcellulose and collagen, novel cell attachment molecules, and/or matrices with growth factors or other signaling molecules embedded within them. This last approach may provide the spatial organization required for replication of complex organ architecture (as reviewed in Saltzman, Nature Medicine 4:272-273, 1998).

### EXAMPLES

The following examples are intended to illustrate but not limit the invention. While they are typical of those that might be used, other procedures known to those skilled in the art may alternatively be used.

### Example 1

The filtration matrix is a fabric made from polyester (DACRON) and the pore size between 20-30 microns. A device is prepared in such a way such that the filtration matrix is provided between an upper chamber containing unfiltered cells and a lower chamber used to collect media and single cells (FIG. 1). A suspension of human ES cells and multicellular ES cell colonies obtained by typsinizing an ES culture is subject to filtration by using vacuum or by centrifugal force produced by a vacuum system or centrifuge, respectively. Following filtration, the filter system is inverted and the multicellular ES cell colonies retained on the filter are efficiently collected by adding culture medium to the previously lower reservoir and passing it across the filtration matrix. The collected multicellular ES cell colonies are inoculated into subsequent cultures. Pluripotent ES cells are propagated therefrom.

### Example 2

In a second embodiment (FIG. 2), the device comprises a hollow, cylindrical filtration matrix, the interior through which the cellular suspension is pumped using a peristaltic pump. The cylinder is itself immersed in a longitudinal chamber through which saline is pumped to collect any cells and medium that passes from the inside of the cylinder to the outside. The pump and flow restriction valve are adjusted such that the flow stream retains desirable multicellular ES cell colonies while single ES cells are pushed through the filter into the outer chamber. The outflow from the interior of the cylinder is enriched in multicellular ES cell colonies.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for isolating or enriching multicellular ES cell colonies from a mixture of multicellular ES cell colonies and single ES cells present in a cellular suspension comprising the steps of:
a. providing a filtration matrix through which single ES cells but not multicellular ES cell colonies can pass from a first side to a second side;
b. contacting the cellular suspension with the first side of the filtration matrix;
c. passing the cellular suspension from the first side to the second side of the filtration matrix; and
d. collecting the multicellular ES cell colonies from the first side of the filtration matrix, thereby isolating or enriching multicellular ES cell colonies therefrom.

2. The method of claim 1 wherein the filtration matrix comprises polyester, polyamide, aramid, acrylic, or PTFE.

3. The method of claim 1 wherein the filtration matrix has a porosity of about 20 microns to about 30 microns.

4. The method of claim 1 wherein the passing is via centrifugation, application of positive pressure to the first side of the filtration matrix, application of negative pressure to the second side of the filtration matrix, or gravity filtration.

5. The method of claim 1 wherein the collecting comprises the steps of:
a. discarding the single ES cells from the second side of the filtration matrix;
b. passing a liquid medium from the second side to the first side of the filtration matrix, whereby multicellular ES cell colonies retained on the first side are suspended in the liquid medium; and
c. collecting the liquid medium.

6. The method of claim 1 wherein the passing is by a combination of lateral flow along the first side of the filtration matrix and transverse flow from the first side to the second side of the filtration matrix.

7. The method of claim 6 wherein the flow is continuous or interrupted.

8. The method of claim 6 wherein the single ES cells pass transversely from the first side to the second side of the filtration matrix, while multicellular ES cell colonies are enriched in the cellular suspension during lateral flow along the first side of the filtration matrix.

9. The method of claim 6 wherein the filtration medium is cylindrical.

10. The method of claim 9 wherein the passing is along the interior of the cylindrical filtration medium, or along the exterior of the cylindrical filtration medium.

11. A method of cultivating euploid human ES cells comprising the steps of:
a. treating a human ES cell culture to detach adherent single ES cells and multicellular ES cell colonies therefrom;
b. collecting a cellular suspension comprising multicellular ES cell colonies and single ES cells from step (a);
c. isolating multicellular ES cell colonies from the suspension by the method of claim 1; and
d. inoculating the multicellular ES cell colonies from step (c) into ES cell culture medium.

12. The method of claim 11 wherein treating is trypsinizing.

13. A device for isolating or enriching multicellular ES cell colonies from a mixture of multicellular ES cell colonies and single ES cells present in a cellular suspension comprising:
a. a filtration matrix through which single ES cells but not multicellular ES cell colonies can pass transversely from a first side to a second side;
b. means for passing the cellular suspension at least transversely through the filtration matrix; and
c. means for collecting the isolated or enriched multicellular ES cell colonies from the first side of the filtration matrix.

14. The device of claim 13 wherein the filtration matrix comprises polyester, polyamide, aramid, acrylic, or PTFE.

15. The device of claim 13 wherein the filtration matrix has a porosity of about 20 microns to about 30 microns.

16. The device of claim 13 wherein the filtration matrix is planar or cylindrical.

17. The device of claim 13 wherein the means for passing comprises a reservoir contiguous with the first side of the filtration matrix and means for collecting comprises a reservoir contiguous with the second side of the filtration matrix.

18. The device of claim 13 further comprising means for passing the cellular suspension laterally along the first side of the filtration matrix.

19. The device of claim 18 wherein the filtration matrix is cylindrical and the first side of the filtration matrix is disposed laterally in the direction of passing of the cellular suspension.

20. The device of claim 19 wherein the first side of the filtration matrix is interior to the cylinder, whereby multiple ES cell colonies are enriched in the cellular suspension passing through the inside of the cylinder, and single ES cells pass transversely across the filtration matrix to the exterior of the cylinder.

21. The device of claim 19 wherein the first side of the filtration matrix is exterior to the cylinder, whereby multiple ES cell colonies are enriched in the cellular suspension passing along the outside of the cylinder, and single ES cells pass transversely across the filtration matrix to the interior of the cylinder.

22. A kit for isolating or enriching multicellular ES cell colonies from a mixture of multicellular ES cell colonies and single ES cells present in a cellular suspension, the kit comprising a filtration matrix through which single ES cells but not multicellular ES cell colonies can pass transversely from a first side to a second side; means for passing the cellular suspension at least transversely through the filtration matrix; means for collecting the isolated or enriched multicellular ES cell colonies; and instructions for use thereof.
